(19) United States
(12) Patent Application Publication
(10) Pub. No.: US 2005/0227372 A1
(43) Pub. Date: Oct. 13, 2005

(54) Diagnostic test kit

(76) Inventor: Waheed N. Khan, Potomac, MD (US)

Correspondence Address: J.W. Gipple;Gipple & Hale, P.O. Box 40513, Washington, DC, 20016, US

(21) Appl. No.: 11/095,133

(22) Filed: Apr. 1, 2005

Related U.S. Application Data
(62) Division of application No. 09/902,966, filed on Jul. 12, 2001, now abandoned.

(51) Int. Cl. C12Q 1/70; G01N 33/558

(52) U.S. Cl. 436/514

(57) ABSTRACT

A diagnostic test kit comprising test strips, extraction instrument, reagent material, all in an elongated tubular container.

### CROSS REFERENCE TO RELATED APPLICATIONS

This application is a division of application Ser. No. 09/902966. The claims 1 through 8 of the present application were claims 12 through 19 of application Ser. No. 09/902966.

### STATEMENT REGARDING FEDERAL SPONSORED RESEARCH AND DEVELOPMENT

None apply to this application.

### FIELD OF INVENTION

The present invention is a diagnostic test kit, all the components of which are fabricated in such minimal dimensions as to fit within a container the approximate size of a ballpoint pen.

### BACKGROUND OF THE INVENTION

There are well known clinical diagnostic procedures that involve collecting specimens (such as blood, fecal matter or urine) which are applied to test strips then dipped into or otherwise subjected to a reagent for purposes of producing a diagnostic reaction. These procedures require clinical conditions and relatively cumbersome equipment. Medical expertise is required to interpret the results.

But frequently the need for such diagnostic testing is in remote places, such as some areas of Africa where the Human Immunodeficiency Virus (HIV) is epidemic, and where clinical facilities are sparse and lacking in sophisticated equipment, as well as medical personnel.

Another problem results from the fact that many diseases (such as HIV) are regarded by some victims of the disease hearing a social stigma, which may result in their being ostracized or subjected to such disadvantages as social and employment discrimination. So people who have reason so suspect they may have such a disease do not want to be tested at a hospital, clinic or other public facility, because they fear that the test results will not remain confidential.

Furthermore, the victims of epidemics such as HIV are frequently poor and cannot afford clinical diagnostic procedures.

Accordingly there is a need for a relatively inexpensive diagnostic test kit, that because of its small size is convenient to distribute in areas lacking conditions for clinical testing and to individuals who can test themselves in private and without risking disclosure of the test results to others.

### DESCRIPTION OF THE PRIOR ART

Applicant's prior U.S. Pat. No. 4,067,776 and the reference cited therein are relevant prior art.

### SUMMARY OF THE INVENTION

The present invention is directed to a diagnostic test kit, which includes an alcohol swab, extracting instrument, a test strip, a reagent liquid, and a set of instructions. All of these components are fitted into a conveniently small container the size and shape of a ball-point pen. The container carries the name and address of the manufacturer.

In the preferred embodiment of the invention, the test strip is contained in a transparent strip holder with only the lower end of the test strip exposed to contact.

In one embodiment of the invention, the extracting instrument is a lancet for pricking a finger to produce a drop of blood.

In other embodiments, the extracting instrument is a swab to collect fecal matter or pipette to collect urine or saliva.

### BRIEF DESCRIPTION OF THE DRAWINGS

FIG. 1 is a top plan view of the container.

FIG. 2 is a side elevational view of the test strip showing three different test phases.

FIG. 3. Is a side elevational view of the lancet extracting instrument.

FIG. 4 is a side perspective view of the reagent liquid in a glass capsule within a buffer tube with flexible walls.

FIG. 5 is a side perspective view of the test strip, properly placed in the buffer tube containing the reagent liquid.

FIG. 6 is a perspective view of the test strip holder.

### DETAILED DESCRIPTION OF THE DRAWINGS

The present invention is directed to a diagnostic test kit, which is relatively inexpensive to produce and because of its small size is convenient to distribute in remote areas lacking conditions for clinical testing and to individuals who can test themselves in private and without risking disclosure of the test results to others.

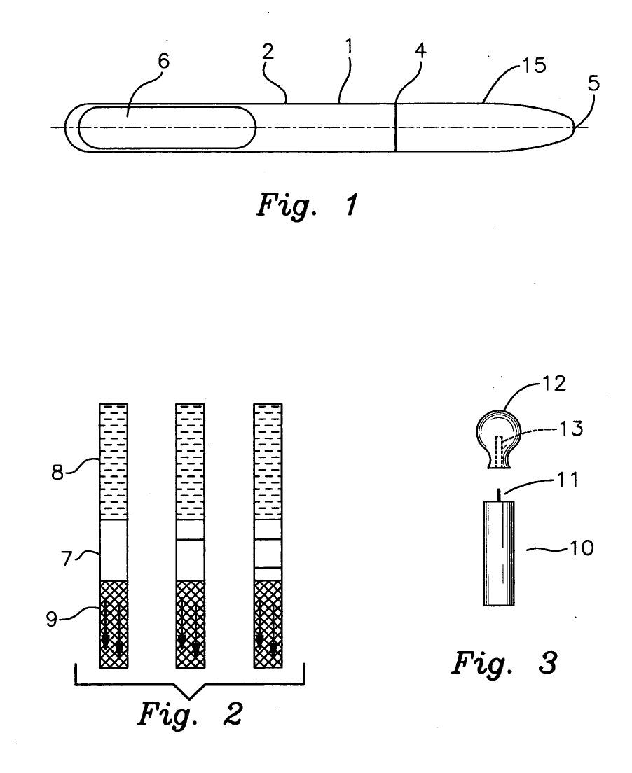

Directing attention to the drawings, FIG. 1 illustrates the container 1, which has a body portion of 2, and a cap portion 3. Cap 3, is detachably connected to body 2, by such conventional means as screw threads, at junction 4. Cap 3 has an orifice 5 on its end opposite to junction 4.

All of the components of the test kit fit within the container 1. The container 1 has a nameplate 6, which in the preferred embodiment also functions as a clip to secure the test kit in a pocket.

The importance of nameplate 6 is that it contains the name and address of the manufacturer or other such source from whom the user can obtain other test kits, in the event that initial test results were not conclusive or if for whatever reasons the user wishes to repeat the test. The diagnostic test kit container is distributed in a package that includes the working mechanism of a ballpoint pen which fits precisely in the container 1, after the testing components thereof have been removed, used and discarded. Thus the container 1 can be easily converted into a usable ball point pen. In third world and underdeveloped countries, a usable ball point pen is valuable and likely to be retained by the user. The user thus retains the nameplate 6 and information as to the source of the diagnostic test kit, if is needed such.

FIG. 2 illustrates the test strip in three different test phases. From left to right, the first phase illustrates a strip with no line across its mid-section, thus indicating that it has not been used, or (if used) that it did not react to the reagent liquid and is invalid. In the second phase, the strip has one line across its mid-section, thus indicating that it has been exposed to the reagent liquid, but that the specimen applied to it carried no antibodies to HIV. The one line is a control to evince that the strip reacted to the reagent liquid and is a valid indicator. In the third stage, the test strip has two lines across its mid-section. The upper line is control, previously described relative to the second phase. The lower line indicates the presence of HIV antibodies in the specimen applied to the test strip.

The test strips illustrated in FIG. 2 each have a mid-section 7, which is the reactive area of the test strip. Each strip has a upper section 8, which is the “handle” or the portion engaged between thumb and finger of the user to dip the strip into the reagent liquid. Each strip has a lower section 9, which is the portion of the test strip to which the specimen is applied.

The test strips referred to above are well known in the art. One such strip is sold by Abbott Labs under the trademark “Determine”.

FIG. 3. illustrates the extracting instrument, which in this preferred embodiment is a lancet, which has a handle portion 10 to support a sharp stainless steel pinpoint 11 which extends from its upper end. A cap 12 fits over handle 10, and has an orifice 13 to receive and contain the pin point 11. In use, the cap is removed and the steel pinpoint 1 is applied to prick a finger tip and draw a drop of blood. The test strip is then pressed lightly to the blood so that some of the blood is absorbed in the lower end 9 of the strip, and moves by capillary action into the reactive mid-section 7 of the strip.

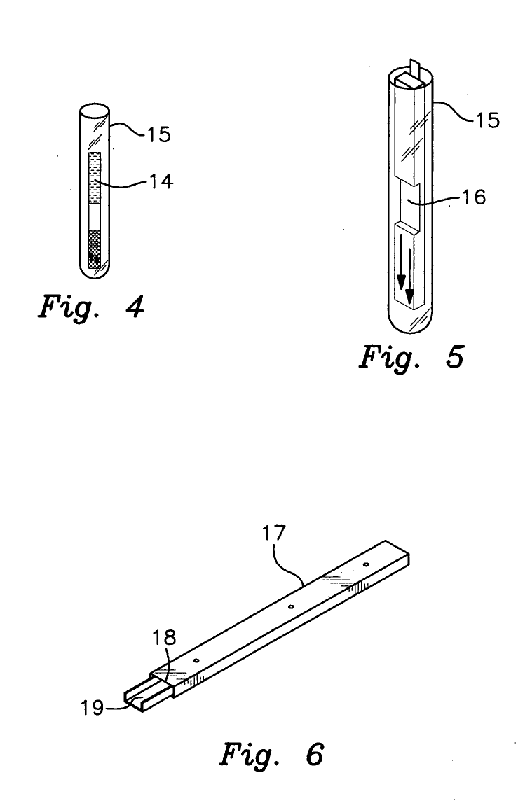

FIG. 4 illustrates the reagent liquid in a breakable glass capsule 14, which is within a flexible plastic buffer tube 15.

In use, the glass capsule 14 is broken within the buffer tube 15 which protects the user's thumb and finger from the glass shards of broken capsule 14. The reagent liquid of capsule 14 is released into the tube 15.

FIG. 5 illustrates the test strip 16 immersed in tube 15.

FIG. 6 illustrates the transparent test strip holder 17, which comprises a narrow, elongated rectangular box, open only at its lower end 18. Extending from the lower end 18 is a tray portion 19. When a test strip is contained in holder 17, the lower end 9 of the test strip (the portion to which the specimen is applied) extends into the open tray portion 19 and is exposed to contact. Thus the specimen can be applied to the lower end 9, but the mid-section 7 and upper section 8 of the test strip are not exposed and therefore not subject to potential contamination. However, because the holder 17 is transparent, the reaction of the test strip to the specimen and reagent can be clearly seen and interpreted.

Other modifications and alternatives to the herein described procedures and components will be apparent to those of ordinary skill in this art and are considered to fall within the scope of the claims defining this invention.

FIG. 4 is a side perspective view of the reagent liquid in a glass capsule within a buffer tube with flexible walls.

FIG. 5 is a side perspective view of the test strip, properly placed in the buffer tube containing the reagent liquid.

FIG. 6 is a perspective view of the test strip holder.

### DETAILED DESCRIPTION OF THE DRAWINGS

The present invention is directed to a diagnostic test kit, which is relatively inexpensive to produce and because of its small size is convenient to distribute in remote areas lacking conditions for clinical testing and to individuals who can test themselves in private and without risking disclosure of the test results to others.

Directing attention to the drawings, FIG. 1 illustrates the container 1, which has a body portion of 2, and a cap portion 3. Cap 3, is detachably connected to body 2, by such conventional means as screw threads, at junction 4. Cap 3 has an orifice 5 on its end opposite to junction 4.

All of the components of the test kit fit within the container 1. The container 1 has a nameplate 6, which in the preferred embodiment also functions as a clip to secure the test kit in a pocket.

The importance of nameplate 6 is that it contains the name and address of the manufacturer or other such source from whom the user can obtain other test kits, in the event that initial test results were not conclusive or if for whatever reasons the user wishes to repeat the test. The diagnostic test kit container is distributed in a package that includes the working mechanism of a ballpoint pen which fits precisely in the container 1, after the testing components thereof have been removed, used and discarded. Thus the container 1 can be easily converted into a usable ball point pen. In third world and underdeveloped countries, a usable ball point pen is valuable and likely to be retained by the user. The user thus retains the nameplate 6 and information as to the source of the diagnostic test kit, if is needed such.

FIG. 2 illustrates the test strip in three different test phases. From left to right, the first phase illustrates a strip with no line across its mid-section, thus indicating that it has not been used, or (if used) that it did not react to the reagent liquid and is invalid. In the second phase, the strip has one line across its mid-section, thus indicating that it has been exposed to the reagent liquid, but that the specimen applied to it carried no antibodies to HIV. The one line is a control to evince that the strip reacted to the reagent liquid and is a valid indicator. In the third stage, the test strip has two lines across its mid-section. The upper line is control, previously described relative to the second phase. The lower line indicates the presence of HIV antibodies in the specimen applied to the test strip.

The test strips illustrated in FIG. 2 each have a mid-section 7, which is the reactive area of the test strip. Each strip has a upper section 8, which is the “handle” or the portion engaged between thumb and finger of the user to dip the strip into the reagent liquid. Each strip has a lower section 9, which is the portion of the test strip to which the specimen is applied.

The test strips referred to above are well known in the art. One such strip is sold by Abbott Labs under the trademark “Determine”.

FIG. 3. illustrates the extracting instrument, which in this preferred embodiment is a lancet, which has a handle portion 10 to support a sharp stainless steel pinpoint 11 which extends from its upper end. A cap 12 fits over handle 10, and has an orifice 13 to receive and contain the pin point 11. In use, the cap is removed and the steel pinpoint 1 is applied to prick a finger tip and draw a drop of blood. The test strip is then pressed lightly to the blood so that some of the blood is absorbed in the lower end 9 of the strip, and moves by capillary action into the reactive mid-section 7 of the strip.

FIG. 4 illustrates the reagent liquid in a breakable glass capsule 14, which is within a flexible plastic buffer tube 15.

In use, the glass capsule 14 is broken within the buffer tube 15 which protects the user's thumb and finger from the glass shards of broken capsule 14. The reagent liquid of capsule 14 is released into the tube 15.

FIG. 5 illustrates the test strip 16 immersed in tube 15.

FIG. 6 illustrates the transparent test strip holder 17, which comprises a narrow, elongated rectangular box, open only at its lower end 18. Extending from the lower end 18 is a tray portion 19. When a test strip is contained in holder 17, the lower end 9 of the test strip (the portion to which the specimen is applied) extends into the open tray portion 19 and is exposed to contact. Thus the specimen can be applied to the lower end 9, but the mid-section 7 and upper section 8 of the test strip are not exposed and therefore not subject to potential contamination. However, because the holder 17 is transparent, the reaction of the test strip to the specimen and reagent can be clearly seen and interpreted.

Other modifications and alternatives to the herein described procedures and components will be apparent to those of ordinary skill in this art and are considered to fall within the scope of the claims defining this invention.

1. A diagnostic test kit for testing material from a living body for indication of a condition present in said body, comprising.
one or more diagnostic test strips for reacting with said body material to indicate said condition;
an instrument for extracting said material from said body and facilitating placement thereof onto a portion of said test strip;
a container of liquid reagent material for reacting on said test strip with said body material when placed thereon to reveal said condition;
an elongated tubular enclosure for holding said test strips extraction instrument and reagent container; said enclosure including a removable closure and an exterior portion for placement of indicia and being of appropriate size to fit in the pocket of human apparel

2. The diagnostic test kit of claim 1 wherein said instrument is a lancet for extracting blood from said human body.

3. The diagnostic test kit of claim 1 wherein said instrument is a swab or pipette.

4. The diagnostic test kit of claim 3 wherein said body material is selected from the group consisting of urine, saliva and fecal matter.

5. The diagnostic test kit of claim 1 wherein said container holding the reagent material is within a second, larger container adapted for receiving one of said test strips and for reaction of the reagent material with said human body material on the said test strip.

6. The diagnostic test kit of claim 1 wherein said test strip includes a first portion proximate either of its opposed ends for gripping the test strip and a second portion proximate the other of the opposed end for receiving the body material and the reagent material, together with indicia present on said second portion for distinguishing from the first portion.

7. The diagnostic test kit of claim 1 wherein said enclosure includes a clip for attachment to the wearing apparel of a human being.

8. The diagnostic test kit of claim 1 wherein said enclosure carries a portion for identifying indicia.
